(19) United States
(12) Patent Application Publication
(10) Pub. No.: US 2026/0041550 A1
(43) Pub. Date: Feb. 12, 2026

(54) VALVE PROSTHESIS HAVING A GRADUAL RELEASE FOR IMPROVED POSITIONING

(71) Applicant: Medtronic, Inc., Minneapolis, MN (US)

(72) Inventors: Barry M. OCONNELL, Galway (IE); Constantin F. Ciobanu, Galway (IE); Patrick King, Gort (IE)

(21) Appl. No.: 18/998,661

(22) PCT Filed: Jul. 14, 2023
(86) PCT No.: PCT/IB2023/057224

§ 371 Date: Jan. 27, 2025

Related U.S. Application Data
(60) Provisional application No. 63/392,533, filed on Jul. 27, 2022.

(51) Int. Cl. A61F 2/24

(52) U.S. Cl. CPC A61F 2/2418; A61F 2220/0025; A61F 2230/0004; A61F 2250/0037

(57) ABSTRACT

A valve prosthesis includes a self-expanding frame and a prosthetic valve coupled to an interior of the frame. The frame includes a plurality of endcrowns at a first end thereof, and a first paddle connected to a first endcrown by a first stem. The first end of the frame is collapsible to a collapsed configuration when a restraining force is applied to the first paddle and to the endcrowns, is configured to expand from the collapsed configuration to a partially deployed configuration when the restraining force is removed from the endcrowns but is still applied to the first paddle, and is configured to expand to a fully deployed configuration when the restraining force is removed from the endcrowns and from the first paddle. A first vale of a lateral dimension of the first end in the partially deployed configuration is at least 70 percent of a second value of the lateral dimension of the first end in the fully deployed configuration.

### FIELD OF THE INVENTION

The present invention is related to a valve prosthesis having a gradual release for improved positioning, and in particular, having a paddle with an elongated stem for gradual release of the valve prosthesis from a delivery device.

### BACKGROUND

Transcatheter heart valve prostheses have been developed for replacing the function of a defective native heart valve. Transcatheter heart valve prosthesis generally include a stent or frame with a prosthetic valve coupled to the frame. The transcatheter heart valve prosthesis is delivered to the native heart valve in a radially compressed configuration such as by a catheter and then is radially expanded or deployed at the native heart valve. Accurate positioning of the heart valve prosthesis is important, as inaccurate position may lead to conduction disturbances, coronary artery obstruction, and/or undesirable paravalvular leakage and/or regurgitation at the implantation site. For example, in a self-expanding heart valve prosthesis, final release of the heart valve prosthesis from the catheter may result in jumping and/or oscillation that may cause the heart valve prosthesis to move from the desired position.

Thus, it is desirable for a heart valve prosthesis to minimize jumping and or oscillation when deployed at the native heart valve to facilitate accurate positioning of the heart valve prosthesis.

### SUMMARY

Embodiments hereof relate to a valve prosthesis which facilitates delivery thereof with improved positioning accuracy.

In a first example, a valve prosthesis includes a self-expanding frame including a plurality of struts forming a plurality of endcrowns at a first end of the self-expanding frame, and a first paddle connected to a first endcrown of the plurality of endcrowns by a first stem. The self-expanding frame is collapsible to a collapsed configuration at the first end of the self-expanding frame when a restraining force is applied to the first paddle and to the plurality of endcrowns. The self-expanding frame is configured to expand from the collapsed configuration to a partially deployed configuration when the restraining force is removed from the plurality of endcrowns but is still applied to the first paddle. In the partially deployed configuration the self-expanding frame has a first value of a lateral dimension at the first end. The self-expanding frame is further configured to expand from the partially deployed configuration to a fully deployed configuration when the restraining force is removed from the plurality of endcrowns and from the first paddle. In in the fully deployed configuration the self-expanding frame has a second value of the lateral dimension at the first end, wherein the first value is at least 70 percent of the second value. The valve prosthesis further includes a prosthetic valve component coupled to an interior of the self-expanding frame.

In a second example, in the valve prosthesis of the first example, the first value of the lateral dimension is in a range of 80 percent to 90 percent of the second value of the lateral dimension.

In a third example, in the valve prosthesis of the first or second example, in the collapsed configuration the plurality of endcrowns store potential energy, and the stored potential energy is reduced by at least 40 percent when the self-expanding frame expands from the collapsed configuration to the partially deployed configuration.

In a fourth example, in the valve prosthesis of the third example, the stored potential energy is reduced by at least 50 percent when the self-expanding frame expands from the collapsed configuration to the partially deployed configuration.

In a fifth example, in the valve prosthesis of any one of the first through fourth examples, a first level of peak kinetic energy is dispersed by the self-expanding frame when expanding from the collapsed configuration to the partially deployed configuration, and a second level of peak kinetic energy is dispersed by the self-expanding frame when expanding from the partially deployed configuration to the fully deployed configuration, and wherein the second level of peak kinetic energy is less than 70 percent of the first level of peak kinetic energy.

In a sixth example, in the valve prosthesis of the fifth example, the second level of peak kinetic energy is in a range of 5 milli-Joule (mJ) to 20 mJ.

In a seventh example, in the valve prosthesis of the sixth example, the second level of peak kinetic energy is in a range of 10 mJ to 15 mJ.

In an eighth example, in the valve prosthesis of any one of the first through seventh examples, the first stem has a length which is at least 4 mm.

In a ninth example, in the valve prosthesis of any one of the first through eighth examples, the first stem is formed as a spring having multiple bends, and the spring is stretched when the self-expanding frame expands from the collapsed configuration to the partially deployed configuration.

In a tenth example, in the valve prosthesis of the ninth example, the spring has a spring constant which is in a range of 0.2 N/mm to 5 N/mm.

In a eleventh example, in the valve prosthesis of any one of the first through tenth examples, with the first end in the radially collapsed configuration, the first stem has at least one bend connecting a first section of the first stem and a second section of the first stem, the first section extending from the first endcrown and the second section extending to the first paddle, wherein a gap between the first section and the second section is less than 1 mm.

In a twelfth example, the valve prosthesis of any one of the first through eleventh examples further comprises a second paddle connected by a second stem to a second endcrown of the plurality of endcrowns, the first paddle and the second paddle being on opposite sides at the first end of the self-expanding frame.

In a thirteenth example, in the valve prosthesis of the twelfth example, the first stem and the second stem have different lengths.

In a fourteenth example, in the valve prosthesis of twelfth example, the first stem and the second stem are about equal in length.

In a fifteenth example, a delivery system comprises a valve prosthesis and a delivery device for percutaneously delivering the valve prosthesis. The valve prosthesis includes a self-expanding frame and a prosthetic valve disposed within the self-expanding frame, the self-expanding frame including a body, a plurality of endcrowns disposed at a first end of the self-expanding frame, a first paddle, and a first stem connecting the first paddle to a first endcrown of the plurality of end crowns. The delivery device includes a sheath configured to restrain the valve prosthesis in a radially collapsed configuration, the sheath being retractable in a proximal direction, and a first shaft for receiving the valve prosthesis thereon. In a delivery configuration of the delivery system the sheath covers the valve prosthesis such that the valve prosthesis is in the radially collapsed configuration and surrounds the first shaft. With the distal end of the sheath retracted to a first position in which the sheath releases the plurality of endcrowns while still restraining the first paddle, the first end of the self-expanding frame of the valve prosthesis is configured to expand from the radially collapsed configuration to a partially deployed configuration such that the first end of the self-expanding frame is radially expanded to a first value of a lateral dimension. With the distal end of the sheath retracted to a second position in which the sheath releases the plurality of endcrowns and releases the first paddle, the first end of the self-expanding frame of the valve prosthesis is configured to expand from the partially deployed configuration to a fully deployed configuration such that the first end of the self-expanding frame is radially expanded to a second value of the lateral dimension, and wherein the first value is at least 70 percent of the second value.

In a sixteenth example, in the delivery system of the fifteenth example, in the radially compressed configuration, the first stem has at least one bend connecting a first section and a second section of the first stem, the first section extending from the first endcrown, and the second section extending to the first paddle.

In a seventeenth example, in the delivery system of sixteenth example, the first stem is configured to cause the first section to push against the second section in the proximal direction during retraction of the sheath.

In an eighteenth example, in the delivery system of the sixteenth example, the delivery device includes a second shaft that surrounds the first shaft and forms or is connected to a prosthesis retention member at a distal end of the second shaft, wherein the prosthesis retention member include a first recessed portion for receiving the first paddle with the self-expanding frame in the delivery configuration or in the partially deployed configuration.

In a nineteenth example, in the delivery system of the eighteenth example, the prosthesis retention member includes a second recessed portion adjacent to the first recessed portion for receiving the at least one bend of the first stem with the self-expanding frame in the delivery configuration.

In a twentieth example, in the delivery system of the eighteenth example, a distal end of the prosthesis retention member includes a protruding portion configured to push against the first section of the first stem in the proximal direction.

### BRIEF DESCRIPTION OF THE DRAWINGS

The accompanying figures, which are incorporated herein, form part of the specification and illustrate embodiments of a delivery system. Together with the description, the figures further explain the principles of and enable a person skilled in the relevant art(s) to make, use, and implant the prosthesis described herein. In the drawings, like reference numbers indicate identical or functionally similar elements.

FIG. 1A illustrates a top view or outflow end view of a valve prosthesis according to an embodiment hereof.

FIG. 1B illustrates a side view of the valve prosthesis of FIG. 1A.

FIGS. 1C and 1D illustrate expanded views of connectors of the valve prosthesis of FIG. 1, according to an embodiment hereof.

FIG. 2A illustrates a side view of a valve prosthesis according to another embodiment hereof.

FIG. 2B illustrates an enlarged view of a connector of the valve prosthesis of FIG. 2A, according to an embodiment hereof.

FIG. 3A schematically illustrates a side view of a delivery system for delivering a valve prosthesis, according to an embodiment hereof.

FIG. 3B schematically illustrates a side view of the delivery system of FIG. 3A with a capsule thereof retracted to expose a valve prosthesis, according to an embodiment hereof.

FIG. 3C schematically illustrates a perspective view of a distal portion of the delivery system with the valve prosthesis in the delivery configuration and an outer not shown for illustrative purposes only.

FIG. 4 schematically illustrates a prosthesis retention member of the delivery system with a paddle of a connector of the heart valve prosthesis disposed therein, according to an embodiment hereof.

FIG. 5 schematically illustrates a prosthesis retention member of the delivery system with a paddle of a connector of the heart valve prosthesis disposed therein, according to an embodiment hereof.

FIGS. 6A and 6B schematically illustrate delivery of a valve prosthesis to a native annulus of an aortic heart valve, according to an embodiment hereof.

FIGS. 7A and 7B schematically illustrate a valve prosthesis partially deployed at a native aortic valve with a restraining force applied to endcrowns of the valve prosthesis, according to an embodiment hereof.

FIG. 8 schematically illustrates a valve prosthesis partially deployed configuration at a native aortic valve with the endcrowns released from a capsule at a distal end of an outer sheath, according to an embodiment hereof.

FIG. 9 schematically illustrates a valve prosthesis in a fully deployed configuration, with endcrowns and paddles released from a capsule at a distal end of an outer sheath, according to an embodiment hereof.

FIG. 10 is a graph illustrating a reduction of strain potential energy as a result of endcrowns moving to reach a partially deployed configuration, according to an embodiment hereof.

FIG. 11 provides a graph illustrating kinetic energy of the endcrowns as they move to reach the partially deployed configuration and then the fully deployed configuration, according to an embodiment hereof.

### DETAILED DESCRIPTION

Specific embodiments of the present invention are now described with reference to the figures, wherein like reference numbers indicate identical or functionally similar elements. Unless otherwise indicated, the terms “distal” and “proximal” when used in the following description for delivery devices refer to a position or direction relative to the treating clinician. “Distal” and “distally” are positions distant from or in a direction away from the clinician, and “proximal” and “proximally” are positions near or in a direction toward the clinician. The terms “distal” and “proximal” when used with respect to a device to be implanted in a vessel refer to the direction of blood flow. Thus, “distal” and “distally” refer to downstream positions or the downstream direction, and “proximal” and “proximally” refer to upstream position or the upstream direction. In addition, the term “self-expanding” is used in the following description with reference to one or more stent structures of the prostheses hereof and is intended to convey that the structures are shaped or formed from a material that can be provided with a mechanical memory to return the structure from a radially compressed or constricted delivery configuration to a radially expanded deployed configuration.

The following detailed description is merely illustrative in nature and is not intended to limit the invention or the application and uses of the invention. Various embodiments of the present disclosure may be used for, e.g., delivering a heart valve prosthesis within a native aortic valve, delivering a heart valve prosthesis within a native mitral valve, delivering a heart valve prosthesis within a native pulmonic valve, delivering a heart valve prosthesis within a native tricuspid valve, delivering a venous valve, or delivering a heart valve prosthesis within a failed previously-implanted prosthesis. Furthermore, there is no intention to be bound by any expressed or implied theory presented in the preceding technical field, background, brief summary or the following detailed description.

Embodiments hereof relate to a valve prosthesis which facilitates delivery thereof with improved positioning accuracy. The valve prosthesis has a first end and a second end, the first end an inflow or proximal to the second end when the valve prosthesis is loaded onto a delivery device. In some instances, the positioning accuracy may be affected by the possibility that, after a clinician has used the delivery device to place the valve prosthesis at a target location, the valve prosthesis may move away from the target location in situ when the first end of the valve prosthesis is released from the delivery device. More specifically, if a high level of potential energy is stored within the first end of the valve prosthesis as the first end is about to be completely released from the delivery device, this high level of potential energy may be converted to a high level of kinetic energy for the first end of the valve prosthesis. The high level of kinetic energy may cause the valve prosthesis to move or tilt when fully deployed or released from the delivery device, which is undesirable. Embodiments hereof relate to a valve prosthesis which includes a connector having a paddle and a stem, wherein the stem allows the first end of the valve prosthesis, or more specifically endcrowns at the first end, to move outward to a partially deployed configuration and expand a size of the valve prosthesis while the paddle has not yet been released from the delivery device. More particularly, the partially deployed configuration may involve the endcrowns moving by at least 70 percent of a distance needed to reach a fully deployed configuration. This movement may reduce how much potential energy remains stored by the endcrowns at the first end of the valve prosthesis when the paddle is about to be released from the delivery device, which would allow the endcrowns to expand the frame from the partially deployed configuration to the fully deployed configuration. The reduced stored potential energy may lead to reduced kinetic energy of the endcrowns as they expand the frame from the partially deployed configuration to the fully deployed configuration. The reduced kinetic energy may reduce a likelihood of the valve prosthesis tilting or otherwise shifting in position after the valve prosthesis is fully deployed, and thus may facilitate accurate placement of a valve prosthesis. Embodiments hereof further relate to a delivery device having a prosthesis retention member which includes a recessed portion and/or a protruding portion for supporting the stem of the connector.

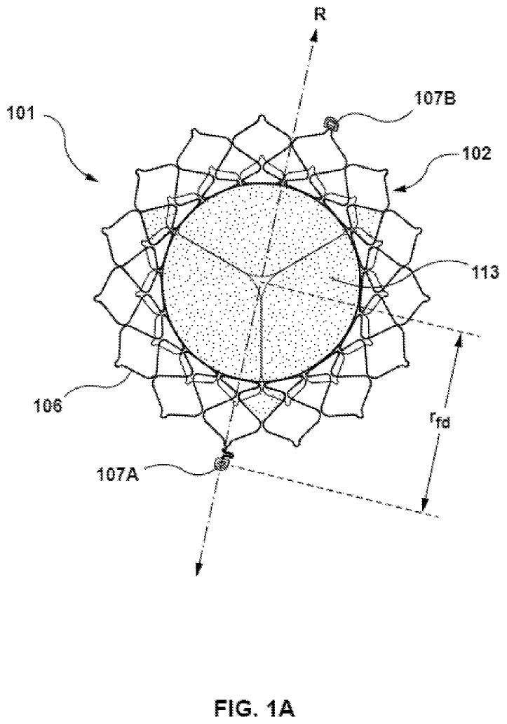

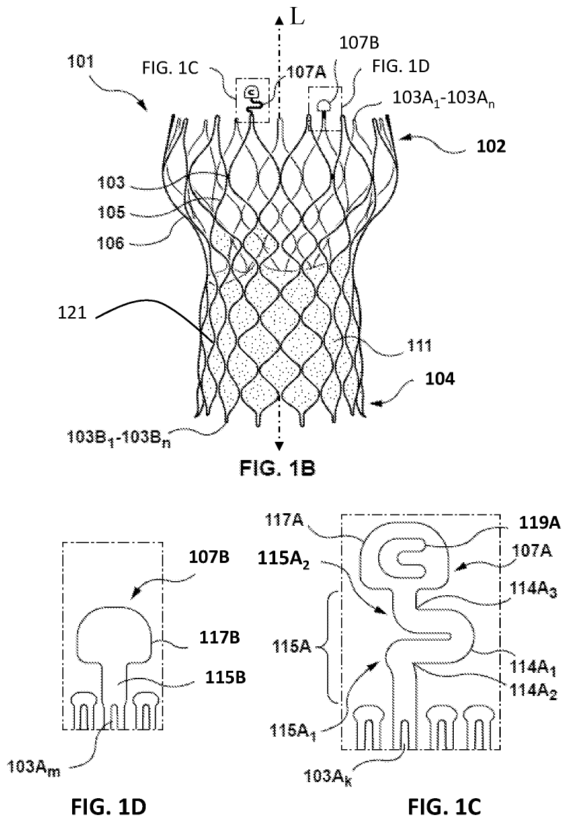

FIGS. 1A-1D illustrate an embodiment of a valve prosthesis 101. In the embodiment of FIGS. 1A-1D, the valve prosthesis is a transcatheter heart valve prosthesis. The valve prosthesis 101 generally includes a self-expanding stent or frame 106 and a prosthetic valve 113 coupled to an interior of the self-expanding frame 106. One skilled in the art will realize that FIGS. 1A-1D illustrate one example of a valve prosthesis and that existing components illustrated in FIGS. 1A-1D may be removed and/or additional components may be added. For example, features described in U.S. Pat. No. 7,914,569 to Nguyen et al. which is herein incorporated by reference in its entirety, may be included in the valve prosthesis 101.

As depicted in FIG. 1B, the self-expanding frame 106 may include a plurality of struts 105 and a plurality of crowns 103 that form a structure having an interior space for the prosthetic valve 113. The plurality of struts 105 may collectively surround the interior of the self-expanding frame 106, and may provide strength or structural support to the frame 106 after the valve prosthesis 101 has been deployed at a treatment site. Each of the crowns 103 may be a curved segment extending between a pair of opposing struts 105. More particularly, the crowns 103 and the struts 105 may form a lattice framework having side openings that are diamond-shaped or form some other shape.

In an embodiment, the self-expanding frame 106 of the valve prosthesis 101 may have a structure that is shaped or formed from a material which can be provided with a mechanical memory to return the structure from a radially collapsed or constricted delivery configuration to a radially expanded deployed configuration. “Self-expanding” as used herein means that frame 106 has a mechanical memory to return to the radially expanded deployed configuration. Mechanical memory may be imparted to the wire or tubular structure that forms frame 106 by thermal treatment to achieve a spring temper in stainless steel, for example, or to set a shape memory in a susceptible metal alloy, such as nitinol, or a polymer, such as any of the polymers disclosed in U.S. Pat. Appl. Pub. No. 2004/0111111 to Lin, which is incorporated by reference herein in its entirety.

FIGS. 1A and 1B depict the valve prosthesis 101 in the radially expanded deployed configuration, or more specifically a fully deployed configuration. The valve prosthesis 101, or more particularly the self-expanding frame 106, may also be collapsible from the deployed configuration back to the radially collapsed configuration. That is, if a restraining force is applied to the self-expanding frame 106 along a radial axis R of the frame 106/valve prosthesis 101, the restraining force may cause the frame 106 of the valve prosthesis 101 to radially collapse to the radially collapsed configuration. As illustrated in FIGS. 1A and 1B, the radial axis R may be an axis which is orthogonal to a longitudinal axis L of the valve prosthesis 101/frame 106. The restraining force may be applied to the frame 106 before and/or during delivery of the valve prosthesis 101, so as to constrain or otherwise collapse the frame 106 of the valve prosthesis 101 and reduce its cross-section for easier movement within a patient's body. Thus, the radially collapsed configuration may also be referred to as a delivery configuration. When the valve prosthesis 101 has been delivered to a treatment site, the restraining force may be removed, and the mechanical memory of the self-expanding frame 106 may cause the frame 106 to expand along the radial axis R to return to the radially expanded deployed configuration.

In an embodiment, when the self-expanding frame 106 of the valve prosthesis 101 is in a deployed configuration, or more specifically a fully deployed configuration, the self-expanding frame 106 may have a generally tubular configuration or other stent configuration used for a valve replacement. In the tubular configuration, the self-expanding frame 106 may have a circular cross-section at a first or outflow end 102 and/or a second or upstream end 104 of the frame 106. In another example, the self-expanding frame 106 may have a cross section with some other shape, such as an ellipsoidal, rectangular, hexagonal, rectangular, square, or other polygonal shape. In the example of FIGS. 1A and 1B, the self-expanding frame 106 may have cross sections that vary in size along the longitudinal axis L of the frame 106/prosthesis 101. In such an example, the self-expanding frame 106 may have a longitudinally asymmetric hourglass configuration, with the first end 102 having an enlarged cross section relative to the second end 104. The valve prosthesis 101 in FIG. 1A may be used, e.g., as an aortic valve replacement, a mitral valve replacement, or some other purpose. In another example, a valve prosthesis of the embodiments herein may include a self-expanding frame having cross sections which are uniform in size along the longitudinal axis L.

FIG. 1A illustrates the valve prosthesis 101, or more specifically the self-expanding frame 106, having a value of r_{fd} for a lateral dimension of the frame 106 at the first end 102 of the frame 106. The lateral dimension may be a dimension which measures a size of the self-expanding frame 106 along the radial axis R, or more specifically measures a size of a particular cross section of the self-expanding frame 106, such as the cross section at the first end 102 of the frame 106. As an example, the lateral dimension may be a width of the self-expanding frame 106. If the self-expanding frame has a cross section which is circular, the lateral dimension may be a radius or diameter of the cross section. In another example, if the cross section is elliptical, the lateral dimension may be a size of a major axis or minor axis of the elliptical cross section.

The prosthetic valve 113 may include valve leaflets which form, e.g., a bicuspid or tricuspid replacement valve which permits blood flow in only one direction. For instance, when the valve prosthesis 101 is deployed at the treatment site, a first end 102 of the valve prosthesis 101 may be an outflow end, while a second end 104 may be an inflow end. After the valve prosthesis 101 is deployed, the prosthetic valve 113 may permit blood flow only in a direction from the inflow end to the outflow end. The valve leaflets of the prosthetic valve 113 may be sutured or otherwise securely and sealingly attached to the interior surface of the frame 106 and/or graft material 111 which encloses or lines the frame 106. Leaflets are attached along their bases to the graft material 111, for example, using sutures or a suitable biocompatible adhesive. Adjoining pairs of leaflets are attached to one another at their lateral ends to form commissures. The orientation of the leaflets within the frame 106 would change depending on which end of the valve prosthesis 101 is the inflow end and which end of the valve prosthesis 101 is the outflow end, thereby ensuring one-way flow of blood through the heart valve prosthesis 101.

In an embodiment, the leaflets of the prosthetic valve 113 may be made of pericardial material; however, the leaflets may instead be made of another material. Natural tissue for replacement valve leaflets may be obtained from, for example, heart valves, aortic roots, aortic walls, aortic leaflets, pericardial tissue, such as pericardial patches, bypass grafts, blood vessels, intestinal submucosal tissue, umbilical tissue and the like from humans or animals. Synthetic materials suitable for use as leaflets include DACRON® polyester commercially available from Invista North America S.A.R.L. of Wilmington, DE, other cloth materials, nylon blends, polymeric materials, and vacuum deposition nitinol fabricated materials. One polymeric material from which the leaflets can be made is an ultra-high molecular weight polyethylene material commercially available under the trade designation DYNEEMA from Royal DSM of the Netherlands. With certain leaflet materials, it may be desirable to coat one or both sides of the leaflet with a material that will prevent or minimize overgrowth. It is further desirable that the leaflet material is durable and not subject to stretching, deforming, or fatigue.

In an embodiment, the graft material 111 may also be a natural or biological material such as pericardium or another membranous tissue such as intestinal submucosa. Alternatively, the graft material 111 may be a low-porosity woven fabric, such as polyester, Dacron fabric, or PTFE, which creates a one-way fluid passage when attached to the stent. In one embodiment, the graft material 111 may be a knit or woven polyester, such as a polyester or PTFE knit, which can be utilized when it is desired to provide a medium for tissue ingrowth and the ability for the fabric to stretch to conform to a curved surface. Polyester velour fabrics may alternatively be used, such as when it is desired to provide a medium for tissue ingrowth on one side and a smooth surface on the other side. These and other appropriate cardiovascular fabrics are commercially available from Bard Peripheral Vascular, Inc. of Tempe, Ariz., for example.

As illustrated in FIG. 1B, the plurality of crowns 103 and struts 105 of the frame 106 may form a series of endmost crowns, also referred to as endcrowns, at the first end 102 and the second end 104 of the self-expanding frame 106. More particularly, a plurality of endcrowns 103A₁-103Aₙ may be formed at the first end 102 of the frame 106, while a plurality of endcrowns 103B₁-103Bₙ may be formed at the second end 104 of the frame 106. The number of endcrowns 103A₁-103Aₙ at the first end 102 and the number of endcrowns 103B₁-103Bₙ at the second end 104 may depend on a size of the valve prosthesis 101 and on its application. In one example, the plurality of endcrowns 103A₁-103Aₙ and the plurality of endcrowns 103B1-103Bₙ may each have between 5-20 endcrowns. When the self-expanding frame 106 is in the radially collapsed configuration, a restraining force may be applied to the frame 106, and hence to the endcrowns 103A₁-103Aₙ and to the endcrowns 103B₁-103Bₙ. When the restraining force is removed from an endcrown (e.g., 103Aₖ) , the mechanical memory of the frame 106 may cause the endcrowns to move in an radially outward direction away from a center of the self-expanding frame 106, so as to expand the frame 106 along the radial axis R.

In the embodiment of FIGS. 1A-1D, the valve prosthesis 101 includes a first connector 107A which extends from one of the plurality of endcrowns 103A₁-103Aₙ at the first end 102 of the frame 106 and a second connector 107B which extends another one of the plurality of endcrowns 103A₁-103Aₙ at the first end 102 of the frame 106. As noted above, the first end 102 of the frame 106 in the embodiment shown is the outflow end. However, in other embodiments, depending on the native heart valve being replaced with the valve prosthesis 101 and/or the delivery path of the delivery device with the valve prosthesis 101 disposed therein, the first and second connectors 107A, 107B may extend from respective ones of the plurality of end crowns at the second end 104 of the frame 106.

FIG. 1C illustrates an expanded view of the first connector 107A. As shown, the first connector 107A extends from a first endcrown 103Aₖ of the plurality of endcrowns 103A₁-103Aₙ. The first connector 107A may provide a connection between at least the first endcrown 103Aₖ and a delivery device. For instance, the first connector 107A may connect at least the first endcrown 103Aₖ to a prosthesis retention member of the delivery device, as described in more detail below, when the valve prosthesis 101 is in the radially collapsed/delivery configuration or in a partially deployed configuration. In the radially collapsed configuration and the partially deployed configuration, a restraining force may be applied to the first connector 107A and may keep the first connector 107A substantially stationary relative to the prosthesis retention member or a capsule/outer sheath of the delivery device. Because the first connector 107A is substantially stationary in this situation, it may limit outward movement of the first endcrown 103Aₖ, which is directly connected to the first connector 107A, and may limit outward movement of neighboring endcrowns that are directly or indirectly connected to the first endcrown 103Aₖ.

In the embodiment of FIGS. 1A-1D, the first connector 107A includes a first paddle 117A and a first stem 115A, as depicted in FIG. 1C. In some instances, the first paddle 117A and/or the first stem 115A may be flat components. The first stem 115A may extend between the first paddle 117A and the first endcrown 103Aₖ, and may connect the first paddle 117A to the first endcrown 103Aₖ. The paddle 117A may be relatively wider, or generally has a larger width, relative to the first stem 115A. The first paddle 117A may have a semi-circular shape, as illustrated in FIG. 1C, or any other shape, such as a circular, rectangular, triangular shape. The first paddle 117A may have a radio-opaque marker 119A, as illustrated in FIG. 1C. In some implementations, the first paddle 117A may have a shape which is able to mate with a recess or other portion of the prosthesis retention member. When the valve prosthesis 101 is in a collapsed/delivery configuration, the first paddle 117A may be mated with the recess of the prosthesis retention member to assist in restraining the self-expanding frame 106 of the valve prosthesis 101 to the collapsed/delivery configuration, and to prevent longitudinal movement of the valve prosthesis within the capsule/outer sheath of the delivery system. When the valve prosthesis 101 or more specifically the self-expanding frame 106 is in the fully deployed configuration, the first paddle 117A may be released from the recess of the prosthesis retention member.

In an embodiment, the mechanical memory of the self-expanding frame 106 may cause the self-expanding frame 106 to store potential energy when a restraining force constrains the frame 106 to the collapsed configuration. The potential energy may be stored at least partially in the endcrowns, including the endcrowns 103A₁-103Aₙ at the first end 102 of the frame 106. When the restraining force is removed, the potential energy may be converted to kinetic energy that expands the self-expanding frame 106 along the radial axis R, which returns the frame 106 to the deployed configuration, such as a fully deployed configuration in which a lateral dimension at the first end 102 of the frame 106/prosthesis 101 has a value of r_{fd}. The expansion of the self-expanding frame 106 may occur through movement of the endcrowns 103A₁-103Aₙ in an outward direction away from a central longitudinal axis of the frame 106, toward respective positions at which the lateral dimension at the first end 102 of the frame 106 has the value of r_{fd}. As discussed above, if the endcrowns 103A₁-103Aₙ have too much kinetic energy when they reach these respective positions associated with the fully deployed configuration, the endcrowns 103A₁-103Aₙ may tend to oscillate around these positions before the kinetic energy dissipates. The oscillations increase a likelihood that the valve prosthesis 101 may shift from a desired implant site or desired implant depth. Similarly, if the endcrowns 103A₁-103Aₙ have too much potential energy, when releases, the valve prosthesis 101 may “jump”, which also may shift the location of prosthesis from the desire implant site or desired implant depth. The shift may increase a chance of valve leakage, pacing issues, and/or valvular pop out.

In an embodiment, the first stem 115A may be configured to limit or reduce how much kinetic energy is in the endcrowns 103A₁-103Aₙ when they reach the respective positions associated with the fully deployed configuration. As discussed below in more detail, the first stem 115A may allow the first end 102 of the valve prosthesis 101, or more specifically the self-expanding frame 106, to expand from the collapsed configuration to a partially deployed configuration that retains less kinetic energy than a connector with a different stem. The partially deployed configuration may provide an intermediate stage between the collapsed configuration and the fully deployed configuration. In the partially deployed configuration, the first paddle 117A is not yet released from the prosthesis retention member, but endcrowns 103A₁-103Aₙ at the first end of the frame 106 are released from the capsule/outer sheath. The first stem 115A may have sufficient slack and/or deformability (which is discussed below) to allow the endcrowns 103A₁-103Aₙ at the first end 102 of the self-expanding frame 106 to move outwardly a substantial amount such as to use a substantial amount of the kinetic energy. More particularly, the first stem 115A may allow the endcrowns 103A₁-103Aₙ in the partially deployed configuration to expand the lateral dimension of self-expanding frame at the first end 102 thereof to at least 70% of a value of the lateral dimension at the first end 102 thereof in the fully deployed configuration. In other words, when the first paddle 117A is not yet released from the prosthesis retention member, the first stem 115A may still allow one or more endcrowns (e.g., 103Aₖ) to move outwardly away from a baseline position associated with the collapsed configuration by a distance which is at least 70% of a total distance separating the baseline position from a final position associated with the fully deployed configuration.

This expansion from the collapsed configuration to the partially deployed configuration may provide an initial release of some of the potential energy stored in the self-expanding frame 106 of the valve prosthesis 101. This initial release of potential energy may reduce a total amount of potential energy still stored by or otherwise remaining in the self-expanding frame 106 in the partially deployed configuration. The reduced amount of stored potential energy in the partially deployed configuration may cause the frame 106 at the endcrowns 103A₁-103Aₙ, including the first endcrown 103Aₖ, to have relatively less kinetic energy when endcrowns 103A₁-103Aₙ are moving toward the respective final positions associated with the fully deployed configuration. The reduced level of kinetic energy in the frame 106 at the endcrowns 103A₁-103Aₙ may reduce or eliminate their oscillation after the endcrowns 103A₁-103Aₙ reach the final positions associated with the fully deployed configuration, which may facilitate accurate placement of the valve prosthesis 101 and reduce positional error.

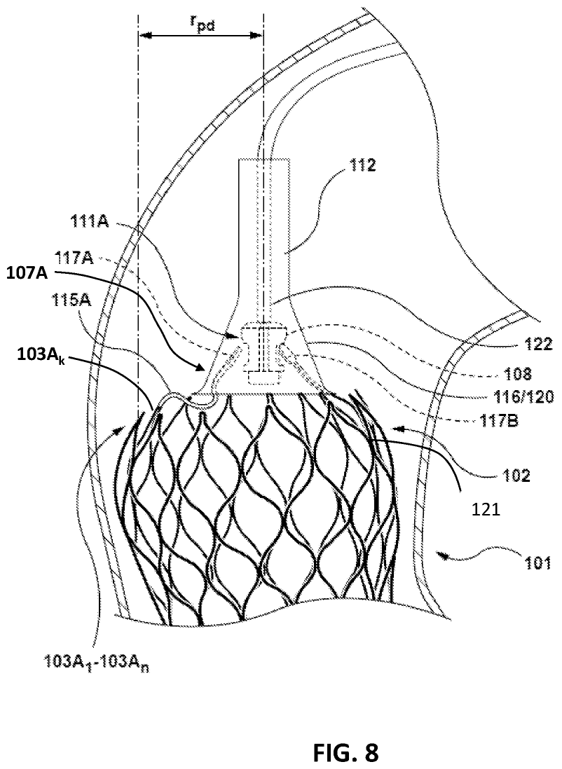

In an embodiment, the first stem 115A may have a length which is sufficiently large to allow at least the first endcrown 103Aₖ to move by a substantial amount even when the first paddle 117A is not released from the prosthesis retention. For example, and not by way of limitation, the first stem 115A may have a length of at least 4 mm. In this embodiment, the length of the first stem 115A or any other stem may refer to the length of the stem when straightened or to an arc length that is measured along a path or curve formed by the stem. As stated above, the first stem 115A may allow the self-expanding frame 106 to reach a partially deployed configuration that is closer to the fully deployed configuration than other stem designs by allowing the endcrowns 103A₁-103Aₙ to expand further radially outwardly before the paddle 117A has been released. For instance, if a restraining force is still applied to the paddle 117A, but is no longer applied to the plurality of endcrowns 103A₁-103Aₙ, the first stem 115A may allow at least the first endcrown 103Aₖ to move outward along the radial axis R by moving away from the paddle 117A, as shown in FIG. 8 described in more detail below.

As further stated above, the first stem 115A may be sufficiently long and/or have sufficient deformability, to allow the self-expanding frame 106 to expand a lateral dimension of the frame 106 at the first end 102 thereof (where the plurality of endcrowns 103A₁-103Aₙ are located) to have a value in the partially deployed configuration which is at least 70 percent of a value of the lateral dimension in the fully deployed configuration. More specifically, the lateral dimension for the self-expanding frame 106 at the first end 102 (where the plurality of endcrowns 103A₁-103Aₙ are located) may have a first value in the partially deployed configuration, and may have a second value in the fully deployed configuration. In this example, the first stem 115A may be sufficiently long and/or have sufficient deformability to allow the first value associated with the partially deployed configuration to be at least 70 percent of the second value associated with the fully deployed configuration. The lateral dimension at the first end 102 of the self-expanding frame 106 may measure how much the first endcrown 103Aₖ has expanded a cross section of the self-expanding frame 106 at the first end 102 thereof. In an embodiment, the lateral dimension at the first end 102 of the frame 106 may be defined by a distance between the first endcrown 103Aₖ and the central longitudinal axis of the frame 106 at the first end 102. In such an embodiment, the first stem 115A may have enough length and/or deformability to allow the first endcrown 103Aₖ to move along the radial axis R by at least 70 percent of a total distance needed to reach the fully deployed configuration. More specifically, if in the fully deployed configuration the first endcrown 103Aₖ is separated from central longitudinal axis at the first end 102 by a distance R_{fd}, the first stem 115A may have a length and/or deformability which is large enough to allow the first endcrown 103Aₖ in the partially deployed configuration to move to a distance of R_{pd} from the central longitudinal axis, wherein R_{pd} is at least 70 percent of R_{fd}.

In an embodiment, the first stem 115A depicted in FIG. 1C may have at least one bend 114A₁ (also referred to as a bended portion) connecting a first section 115A₁ and second section 115A₂ of the first stem 115A when the first end 102 of the frame 106 is in the radially compressed configuration. In a more specific embodiment, the first stem 115A may include multiple bends 114A₁-114A₃. The bends 114A₁-114A₃ may allow a relatively long stem to fit into a relatively short longitudinal distance between the end crowns 103A₁-103Aₙ and the small prosthesis retention member. As stated above, a longer stem may provide more slack for endcrowns (e.g., 103Aₖ) to move outward when the frame 106 is in the partially deployed configuration when the first paddle 117A is still restrained, wherein such movement may release some of the stored potential energy at the endcrowns, which in turn reduces an amount of kinetic energy at the endcrowns after the first paddle 117A is released.

In some instances, the at least one bend 114A₁, or more specifically the multiple bends 114A₁-114A₃, may form the first stem 115A as a spring which is stretchable when the self-expanding frame 106 is expanded from the collapsed configuration to the partially deployed configuration. The bends 114A₁-114A₃ may provide the first stem 115A with stretchability, or more generally deformability. The deformability may refer to an ability of the spring to undergo elastic deformation, while the stretchability may refer to an ability of the spring to be stretched. Stretching of the spring refers to a deformation which decreases a degree of curvature at the bends 114A₁-114A₃. More particularly, a bend such as 104A₁ may be a curved portion that provides a pivot point connecting adjacent sections of the first stem 115A, such as 115A₁ and 115A₂. When the spring is stretched, the adjacent sections 115A₁ and 115A₂ may be pulled further apart from each other, though the two sections 115A₁ and 115A₂ may still remain connected at the pivot point. Further, the stretching of the spring may decrease a degree of curvature of this curved portion. Thus, the bends 114A₁-114A₃ may configure the first stem 115A as a stretchable spring. In such instances, the first paddle 117A connected to the first stem 115A may be referred to as a spring paddle. The spring formed by the first stem 115A may have a spring constant which is in a range of 0.2 ro 5N/mm (e.g., a spring constant of 1 N/mm). However, this is not meant ot be limited, and other values may be used.

In some instances, the at least one bend 114A₁ may leave only a small gap between the first section 115A₁ and the second section 115A₂, as illustrated in FIG. 1C. The small gap may be, e.g., equal to or less than 0.1 mm. The limited gap may facilitate deployment of the valve prosthesis 101, by preventing too much movement of the stem 115A when a sheath is being retracted past the stem 115A, as discussed below in more detail.

A valve prosthesis of the embodiments herein may include only a single connector, exactly two connectors, or some other number of connectors (e.g., exactly three connectors, or at least four connectors). The prosthesis retention member may have the same number of recesses as the number of connectors, wherein each of the recesses corresponds to one of the connectors. In some instances, the connectors may be equally circumferentially spaced around the first end 102 of the self-expanding frame 106 of the valve prosthesis 101. In the embodiment of FIGS. 1A-1D, the valve prosthesis 101 includes the first connector 107A and further includes a second connector 107B. An expanded view of the second connector 107B is provided in FIG. 1D. The first connector 107A may extend from a first endcrown 103Aₖ, while the second connector 107B may extend from a second endcrown 103Aₘ. The second connector 107B may include a second paddle 117B and a second stem 115B that connects the second paddle 117B to the second endcrown 103Aₘ. In this embodiment, the first connector 107A and the second connector 107B may be disposed at opposite sides of a cross section at the first end 102 of the self-expanding frame 106 at which the plurality of endcrowns 103A₁-103Aₘ are located, i.e., 180 degrees apart around the circumference of the frame 106. As discussed below, the first connector 107A and the second connector 107B may be configured to be mated with two respective recesses of a prosthesis retention member. In the embodiment of FIGS. 1A-1D, the stem 115A is longer than the stem 115B and the stem 115A includes bends 114A₁-114A₃ while stem 115B does not include bends. Thus the stem 115B be may be substantially parallel to the central longitudinal axis of the frame 106 when the frame 106 is in the radially expanded configuration.

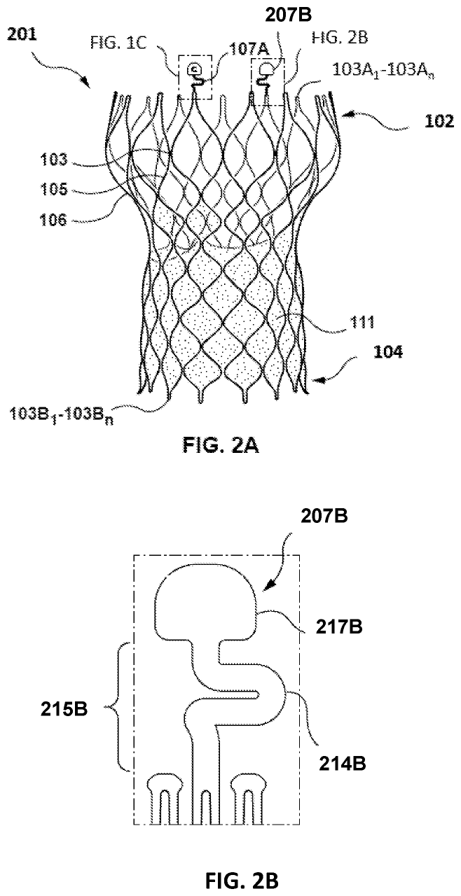

In another embodiment, a valve prosthesis of the embodiments herein may include a combination of connectors that each has at least one bend, or more specifically, are each configured as a spring paddle. For instance, FIGS. 2A-2B depict a valve prosthesis 201 which includes the first connector 107A of FIG. 1C, and further includes a second connector 207B having a paddle 217B and a stem 215B connecting the paddle 217B to one of the endcrowns 103A₁-103Aₙ. The second connector 207B is depicted in FIG. 2B, and may replace the connector 107B, or may be present in addition to the connector 107B. Like the stem 115A of the first connector 107A, the stem 215B of the second connector 207B has at least one bend 214B. In the example of FIGS. 2A-2B, the first connector 107A has a radio-opaque marker 106A, while the connector 207B may have no radio-opaque marker. In some instances, the first connector 107A and the second connector 207B may have the same structure and/or same size. For example, the stem 115A and the stem 215B may have the same shape and/or the same length. In other instances, the first connector 107A and the second connector 207B may have different structures and/or different sizes. For example, the stem 115A of the first connector 107A and the stem 215B of the second connector 207B may have different lengths.

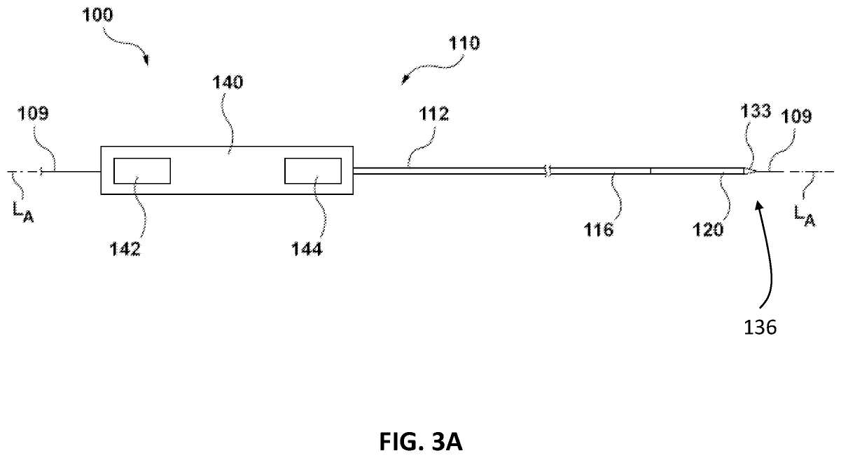

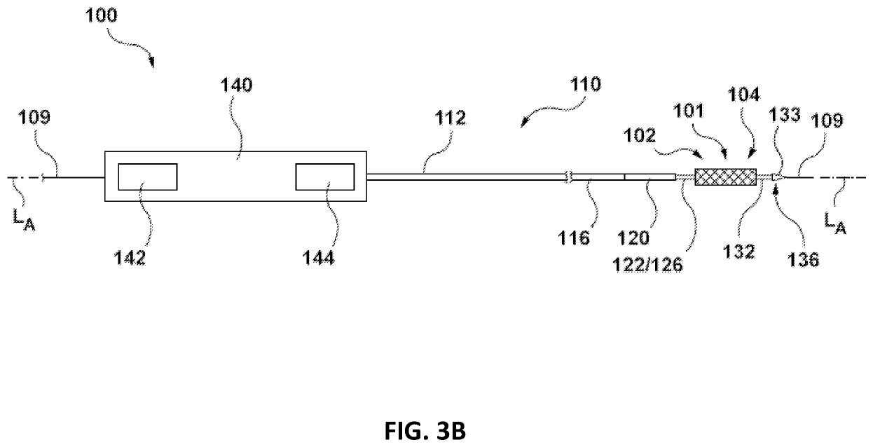

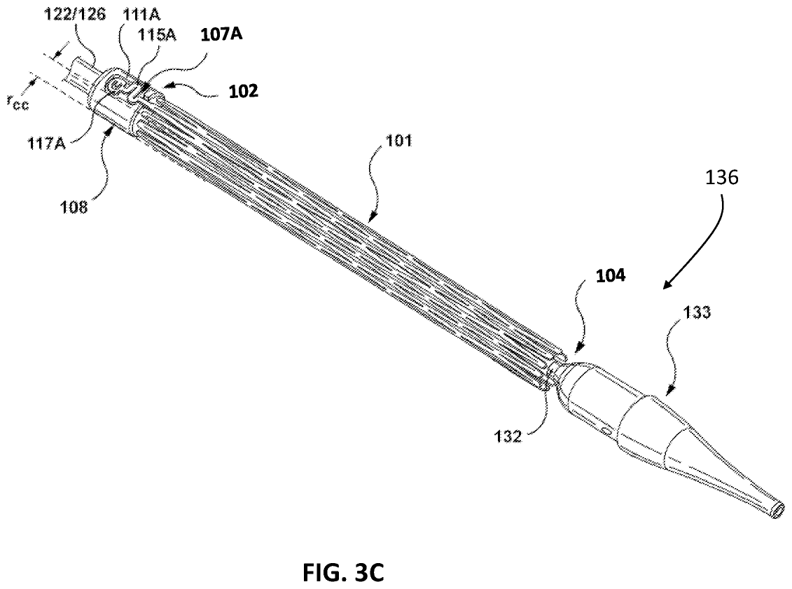

As stated above, a valve prosthesis (e.g., 101/201) of the embodiments herein may be delivered to a treatment site via a delivery device, wherein the delivery device and the prosthesis may form a delivery system. FIGS. 3A and 3B illustrate a side view of a delivery system 100 that includes the valve prosthesis 101 (see FIG. 3B) and a delivery device 110 configured to percutaneously deliver the valve prosthesis 101 to a treatment site. The delivery device 110 may include a shaft 132 on which the valve prosthesis 101 is mounted, and may include a sheath 112 for restraining the valve prosthesis 101 to the collapsed configuration and/or the partially deployed configuration. FIG. 3A illustrates a configuration in which a capsule 120 is coupled to or formed by a distal end 116 of the sheath 112 and is covering the valve prosthesis 101 and the shaft 132, while FIG. 3B illustrates a configuration in which the capsule 120 is retracted to a position at which the valve prosthesis 101 is exposed.

As illustrated in FIGS. 3A and 3B, the delivery device 110 may include a handle having at least one actuator, such as a first actuator 142 and a second actuator 144. The first actuator 142 may be configured to advance the shaft 132 distally from an insertion site to a treatment site within a patient's body (e.g., via a guidewire 109). The shaft 132 (also referred to as an inner shaft 132) may have a proximal end which terminates within the handle 140, and have a distal end 136. As stated above, the valve prosthesis 101 may be mounted at the distal end of the shaft 132 during delivery, and may be restrained to a delivery configuration, or more specifically a radially collapsed configuration, by the sheath 112 that is covering the valve prosthesis 101. In such a situation, the distal end 136 of the shaft 132 may be disposed within the sheath 112. More specifically, the distal portion 116 of the sheath 112 may define or may be coupled to a capsule 120, which may be configured to compressively retain the valve prosthesis 101 in the radially collapsed configuration so as to facilitate delivery to a desired treatment site. Because the sheath 112 may surround the shaft 132 and the valve prosthesis 101, the sheath 112 may also be referred to as an outer sheath. Further, because the shaft 132 is surrounded by the valve prosthesis 101 and the sheath 112, the shaft 132 may be referred to as an inner shaft.

The second actuator 144 may be configured to manipulate the sheath 112, such as by sliding the sheath 112 (and hence the capsule 120) relative to the shaft 132 and the valve prosthesis 101, in a direction parallel to a longitudinal axis LA of the shaft 132, so as to retract the sheath 112 in a proximal direction. More particularly, the second actuator 144 is coupled to the outer sheath 112, and is generally constructed to provide selective proximal retraction and distal advancement of the outer sheath 112, and the capsule 120 attached thereto, relative to the valve prosthesis 101 held in a radially compressed, delivery configuration therein for covering and uncovering the valve prosthesis 101. The second actuator 144 may assume any construction that is capable of providing the desired sheath actuation functionality, such as those described in U.S. Pat. No. 8,579,963 to Tabor, which is assigned to the same assignee as the present disclosure and which is herein incorporated by reference in its entirety. Although the outer sheath 112 is described herein as a single component, this is not meant to limit the design, and the outer sheath 112 may include components such as, but not limited to a proximal shaft or other components suitable for the purposes described herein. The handle 140 can have any shape or size appropriate for convenient handling by a user.

FIG. 3C is a perspective view of a distal portion of the delivery system 100 but with the outer sheath 112 not shown for illustrative purposes only. As illustrated in FIG. 3C, the delivery system 100 may include a distal tip 133 or a tapered flexible nosecone coupled to the distal end 136 of the shaft 132. The inner shaft 132 may define a lumen that encloses the guidewire 109, such that the delivery system 100 may be slidingly disposed and tracked over the guidewire 109. FIG. 3C further illustrates a lateral dimension of the valve prosthesis 101/self-expanding frame 106 at the first end 102 having a value of r_{cc} when the prosthesis 101/frame 106 is in the collapsed configuration.

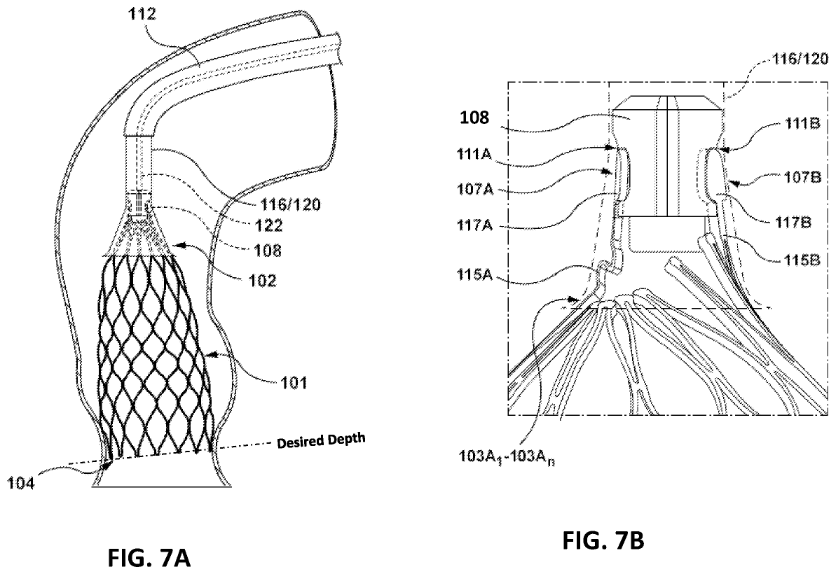

In the embodiment of FIGS. 3B and 3C, the shaft 132 may be coupled to a pusher shaft or middle member 122 at a prosthesis retention member 108. The pusher shaft 122 may be concentrically disposed over the inner shaft 132, and may have a proximal end which terminates within the handle 140 and a distal end 126 disposed inside of the outer sheath 112 when the outer sheath 112 is disposed over the valve prosthesis 101. The distal end 126 of the pusher shaft 122 may include the prosthesis retention member 108 that is configured to connect to or otherwise engage with at least the connector 107A or other component of the valve prosthesis 101. In an embodiment, the prosthesis retention member 108 may be or may include a spindle which is releasably coupled to a first end (e.g., 102) of the valve prosthesis 101. The spindle may be a tubular component having at least a first recessed portion 111A formed on an outer surface thereof. More specifically, in this embodiment, although not shown in FIG. 3C, the prosthesis retention member includes the first recessed portion 111A and a second recessed portion 111B (see FIG. 7B) for retaining the first connector 107A and the second connector 107B/207B, respectively.

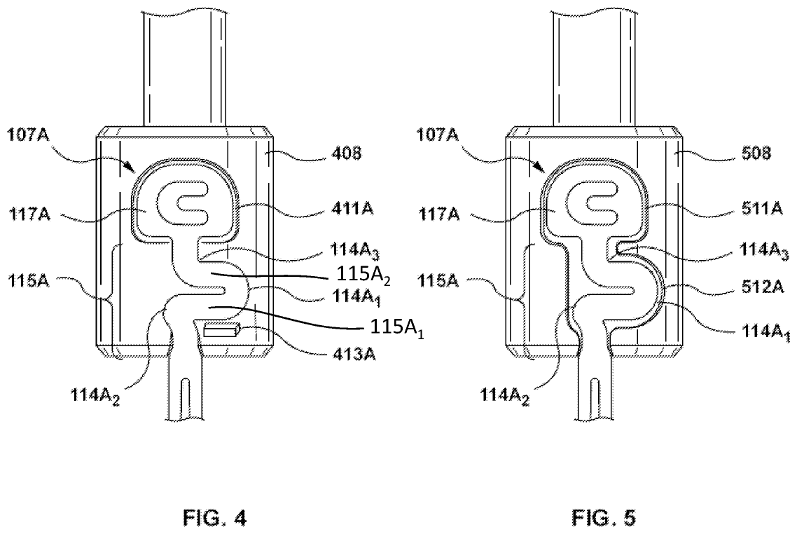

In the embodiment shown, the first recessed portion 111A of the prosthesis retention member 108 is configured to receive the paddle 117A and/or the stem 115A of the connector 107A. More specifically, the first recessed portion 111A may be configured to fit around the paddle 117A and/or the stem 115A, so as to allow the paddle 117A and/or stem 115A to mate with the first recessed portion 111A. For example, FIG. 4 illustrates a prosthesis retention member 408, or more particularly a spindle, which may be an embodiment of the prosthesis retention member 108. The prosthesis retention member 408 has a first recessed portion 411A. When the valve prosthesis 101 is in the delivery configuration or in the partially deployed configuration, the first connector 107A of the prosthesis 101 may be connected to the prosthesis retention member 408. In such configurations, the first paddle 117A may disposed within the first recessed portion 411A. More particularly, the first recessed portion 411A may fit around the first paddle 117A, such that the first paddle 117A is mated to the first recessed portion 411A.

In the embodiment of FIG. 4, the prosthesis retention member 408 may further include a protruding portion 413A which protrudes from a surface thereof. The protruding portion 413A may be disposed adjacent the first second 115A₁ of the first stem 115A to substantially align the paddle 117A with a base of the spring, i.e., substantially align the paddle the bends 114A₂ and 114A₃ in the stem 115A. The protruding portion 413A may act as a bracing protrusion that provides support for the first stem 115A at a base thereof.

FIG. 5 depicts another embodiment of a prosthesis retention member 508, or more specifically a spindle, which may be an embodiment of the prosthesis retention member 108. The prosthesis retention member 508 has a first recessed portion 511A and a second recessed portion 512A adjacent to the first recessed portion 511A. When the valve prosthesis 101 is in the delivery configuration or the partially deployed configuration, the first recessed 511A may receive the first paddle 117A, while the second recessed portion 512A may receive the first stem 115A. More particularly, the first recessed portion 115A may fit around the first paddle 117A of the connector 107A, such that the first paddle may be mated with the first recessed portion 115A. The second recessed portion 512A may fit around a shape of the first stem 115A, including a bend 114A₁ of the first stem 115A, so that the first stem 115A may be mated with the second recessed portion 512A.

In an embodiment, the prosthesis retention member 408/508 may include multiple recessed portions for mating with multiple paddles of multiple connectors of a valve prosthesis, wherein the prosthesis retention member 408/508 may include a respective recessed portion for each connector. For instance, the recessed portion 411A/511A may be located on one side of the prosthesis retention member 408/508 and configured to mate with the paddle 117A of the connector 107A. The prosthesis retention member 408/508 may further include a second recessed portion on an opposite side of the first recessed portion 411A/511A to mate with another paddle, such as the paddle 117B of the connector 107B of FIG. 1D, or the paddle 217B of the connector 207B of FIG. 2B. As explained above, FIG. 7B shows a second recessed 111B, which could be the recessed portion 411A or the recessed portion 511A.

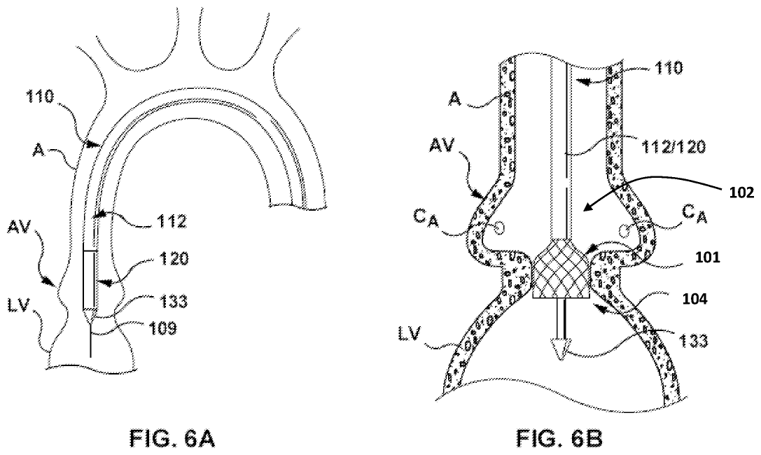

As stated above, a valve prosthesis and delivery device of the embodiments herein may facilitate accurate delivery and deployment to a treatment site. FIGS. 6A through 9 illustrate an example method of delivery and deployment of the valve prosthesis 101 by the delivery system 100. The delivery and/or deployment may be part of, e.g., an interventional cardiology and/or interventional radiology procedure, in which the delivery system 100 including the delivery device 110 is transluminally advanced in a retrograde approach through the vasculature to the treatment site, which in this instance is a target diseased native aortic valve AV that extends between a patient's left ventricle LV and a patient's aorta A. Delivery of the delivery system 100 to the native aortic valve AV is accomplished via a percutaneous transfemoral approach in which the delivery system is tracked through the femoral artery, up the aorta and around the aortic arch in order to access the native aortic valve AV. The delivery system 100 may also be positioned within the desired area of the heart via different delivery methods known in the art for accessing heart valves, for example, via a direct aortic delivery method, or a subclavian artery delivery method.

In an embodiment, the delivery system 100 is tracked over the guidewire 109 that has previously been inserted into the patient vasculature. During delivery, the valve prosthesis 101 remains compressed within the capsule 120 of the outer sheath 112 as the delivery system 100 is manipulated and navigated through the vasculature. For instance, FIG. 6A illustrates the capsule 120 at a distal end of the sheath 112 covering the valve prosthesis 101, wherein the capsule 120 radially compresses or otherwise restrains the valve prosthesis 101 to the radially collapsed configuration. The delivery system 100 is advanced until the distal tip 133 thereof is distal to the native aortic valve AV and disposed within the left ventricle LV as shown in FIG. 6A, such that the second end 104 of the prosthesis 101 (which is the inflow end when the valve prosthesis 101 is configured for placement in a native aortic valve) is positioned at an annulus of a native aortic heart valve, as illustrated in FIG. 6B.

FIG. 6B presents a sectional view of the native aortic heart valve AV, and depicts the second end 104 of the valve prosthesis 101 deployed at the annulus of the native aortic heart valve AV by proximal retraction of the outer sheath 112 and the capsule 120. The outer sheath 112 and the capsule 120 are proximally retracted via the second actuator 144 of the handle 140. The configuration depicted in FIG. 6B is a partially deployed configuration, but may also be referred to as a collapsed configuration at the endcrowns 103A₁-103Aₙ at the first end 102 of the frame 106 since a restraining force is still applied to endcrowns 103A₁-103Aₙ at the first end 102 (e.g., outflow end) of the valve prosthesis 101. FIGS. 7A and 7B depict the valve prosthesis 101 after the capsule 120 at the distal end 116 of the sheath 112 has been further retracted relative to the amount of retraction in FIG. 6B. In FIGS. 7A and 7B, the valve prosthesis 101/frame 106 is in a partially deployed configuration with more of the valve prosthesis 101/frame 106 radially expanded, but the frame 106 at the endcrowns 103A₁-103Aₙ at the first end 102 of the frame 106 remain in a radially collapsed configuration since a restraining force from the capsule 120 is still applied to endcrowns 103A₁-103Aₙ at the first end 102. As depicted in FIG. 7B, the first paddle 117A may be mated to the recessed portion 111A of the prosthesis retention member 108 and the second paddle 117B may be mated to the recessed portion 111B of the prosthesis retention member 108. Further the capsule 120 may be applying the restraining force further to the paddles 117A, 117B and the stems 115A, 115B of the connectors 107A, 107B. Still further, as shown in FIG. 7B, capsule 120 is applying a restraining force to the endcrowns 103A₁-103Aₙ and the first end 102 of the valve prosthesis 101, thereby maintaining the endcrowns 103A₁-103Aₙ in the radially collapsed configuration.

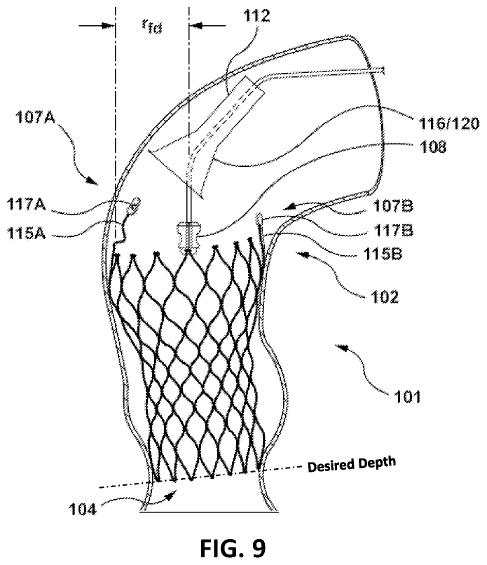

As the outer sheath 112 and the capsule 120 continue to be proximally retracted, the capsule 120 no longer surrounds the endcrowns 103A₁-103Aₙ, but continues to surround the paddles 117A, 117B disposed in the recessed portions 111A, 111B of the retention member 108, as shown in FIG. 8. Thus, in this partially deployed configuration, the outer sheath 112/capsule 120 is no longer applying a restraining force to the plurality of endcrowns 103A₁-103Aₙ. Accordingly, the restraining force is removed from the endcrowns 103A₁-103Aₙ, thus releasing them from the outer sheath 112/capsule 120. Thus, the endcrowns 103A₁-103Aₙ of the valve prosthesis 101 may move outward due to the mechanical memory of the self-expanding frame 106. However, because the outer sheath 112/capsule 120 still covers the paddles 117A, 117B, and the paddles 117A, 117B are each connected to one of the endcrowns 103A₁-103Aₙ, the endcrowns 103A₁-103Aₙ cannot fully expanded. Thus, in the embodiment of FIG. 8, the endcrowns 103A₁-103Aₙ are in a partially deployed configuration. When the capsule 120 is further retracted to uncover and release the paddles 117A, 117B, the restraining force is no longer applied to the paddles 117A, 117B, which may allow the valve prosthesis 101 to expand to the fully deployed configuration, which is illustrated in FIG. 9.

As stated above, one aspect of the present application relates to reducing a level of kinetic energy in the endcrowns 103A₁-103Aₙ when they are reaching respective positions associated with the fully deployed configuration. The kinetic energy in an endcrown, such as the first endcrown 103Aₖ, may be created when the paddle 117A is released due to retraction of the capsule 120. As stated above, the kinetic energy may be created from release of potential energy stored by the endcrown 103Aₖ as a result of the mechanical memory of the self-expanding frame 106. If a relatively high amount of potential energy is stored by the endcrown 103Aₖ when the valve prosthesis is in the partially deployed configuration, then the relatively high amount of potential energy may be converted to a relatively high level of kinetic energy when the paddle 117A is released and the endcrown 103Aₖ and neighboring endcrowns are allowed to expand the frame 106 toward the fully deployed configuration. Thus, as further discussed above, the first stem 115A may be configured to provide an initial release of the stored potential energy in the endcrown 103Aₖ and neighboring endcrowns in the partially deployed configuration, which may reduce the resulting kinetic energy in the endcrowns when they are moving toward the fully deployed position.

In an embodiment, the first stem 115A may provide the initial release of potential energy by allowing the endcrown 103Aₖ and neighboring endcrowns to move outward to expand the frame 106 when the frame 106 is still in the partially deployed configuration. More specifically, the first stem 115A may provide sufficient slack and/or deformability (or, more specifically, stretchability) to allow the first endcrown 103Aₖ and neighboring endcrowns to move outwardly away from the paddle 117A so as to radially expand the first end 102 to the partially deployed configuration. As stated above, the first stem 115A may include at least one bend which increases its total length, wherein the increased length provides the slack to enable radial expansion of the first end of the frame 106 to the partially deployed configuration with a larger cross-sectional dimension than the same frame 106 with a shorter first stent. The expansion may increase a lateral dimension of the valve prosthesis 101 at the plurality of endcrowns 103A₁-103Aₙ at the first end 102 of the frame 106 in the partially deployed configuration to a first value r_{pd}. As further discussed above, the first stem 115A may cause the first value r_{pd} associated with the partially deployed configuration to be at least 70 percent of a second value r_{fd} of the lateral dimension in a fully deployed configuration. In some instances, the first stem 115A may allow the first value r_{pd} of the lateral dimension associated with the partially deployed configuration to be in a range between 80 percent to 90 percent of the second value r_{fd} of the lateral dimension of the self-expanding frame associated with the fully deployed configuration.

Another way to describe the partially deployed configuration of the endmost crowns 103A₁-103Aₙ is via expansion of a node of the frame adjacent the endmost crowns 103A₁-103Aₙ. As described above, the frame 106 includes endmost crowns at each end of the frame. Further, the location where adjacent rows of struts 105 and crowns 103 of the frame 106 meet may be referred to as nodes 121 (see FIG. 1 and FIG. 8). As shown in FIG. 8, the frame 106 at the node 121 adjacent the endmost crowns 103A₁-103Aₙ radially expands as the capsule 120 is proximally retracted. Further, the more the endmost crowns 103A₁-103Aₙ radially expand prior to final release, the more the frame at the node 121 adjacent the endmost crowns 103A₁-103Aₙ radially expands. Thus, in an embodiment, the frame 106 in the partially expanded configuration with the endmost crowns 103A₁-103Aₙ released from the capsule 120 but the paddles still restrained by the capsule, a cross-sectional dimension such as a diameter or radius of the frame 106 at the node 121 adjacent the endmost crowns 103A₁-103Aₙ is at least 80% or at least 85% the cross-sectional dimension at the node 121 adjacent the endmost crowns 103A₁-103Aₙ in the fully deployed configuration.

This expansion of the frame 106 to the partially deployed configuration releases some of the potential energy stored by the frame 106, including potential energy stored by at the endcrowns 103A₁-103Aₙ. Thus, when the capsule 120 is retracted in the proximal direction past the paddles 117A/117B and the prosthesis retention member 108, the endcrowns 103A₁-103Aₙ may move toward the fully deployed configuration of FIG. 9 with only a limited amount of kinetic energy. As a result, the endcrowns 103A₁-103Aₙ may exhibit no oscillation or reduced/limited oscillation when they reach positions associated with the fully deployed configuration. The lack of oscillation/limited oscillation may better ensure that the valve prosthesis 101 does not shift away from a desired depth in the annulus of the native aortic heart valve. Further, with less potential energy stored at the endcrowns 103A₁-103Aₙ prior to final deployment, there is less of a tendency for the first end 102 of the frame to “jump” when the paddles 117A/117B are released from the capsule 120.

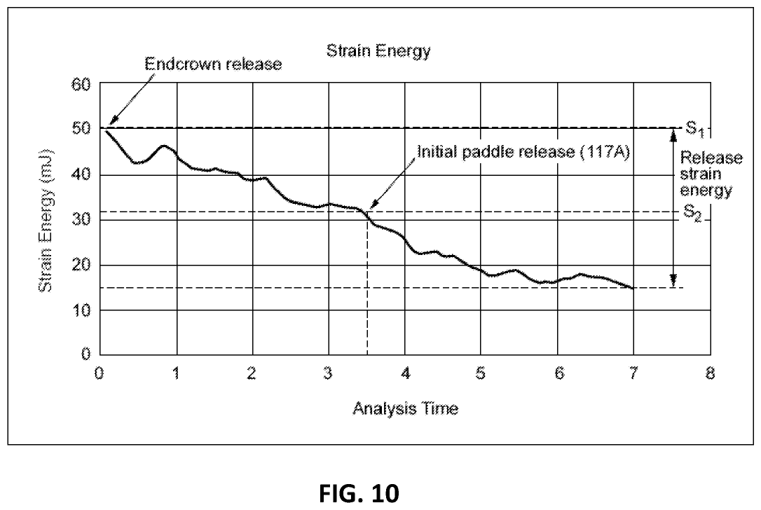

As stated above, the self-expanding frame 106 of the valve prosthesis 101 in the collapsed configuration may store potential energy, and expansion of the self-expanding frame 106 from the collapsed configuration to the partially deployed configuration may release a portion of the stored potential energy. Further, the stem 115A of the connector 107A may have a length which permits a substantial amount of expansion to occur, which may allow a large portion of the stored potential energy to be expended by the expansion from the collapsed configuration to the partially deployed configuration. For instance, FIG. 10 provides a graph which illustrates an amount of strain potential energy stored by the self-expanding frame 106 of the valve prosthesis 101. In this example, when the endcrowns 103A₁-103Aₙ are released by the capsule 120, the frame 106 may be storing about 50 mJ of strain potential energy. When the capsule 120 retracts proxially to release the endmost crowns 103A₁-103Aₙ therefrom but still maintains the paddles 117/A/117B restrained, such as in FIG. 8, the strain potential energy is reduced by about 40 percent, from about 50 mJ to about 30 mJ. In some instances, the strain potential energy may be reduced by at least 50 percent. This reduction in the stored potential energy may lead to a reduction in the kinetic energy of the endcrowns 103A₁-103Aₙ as they expand the frame 106 to the fully deployed configuration.

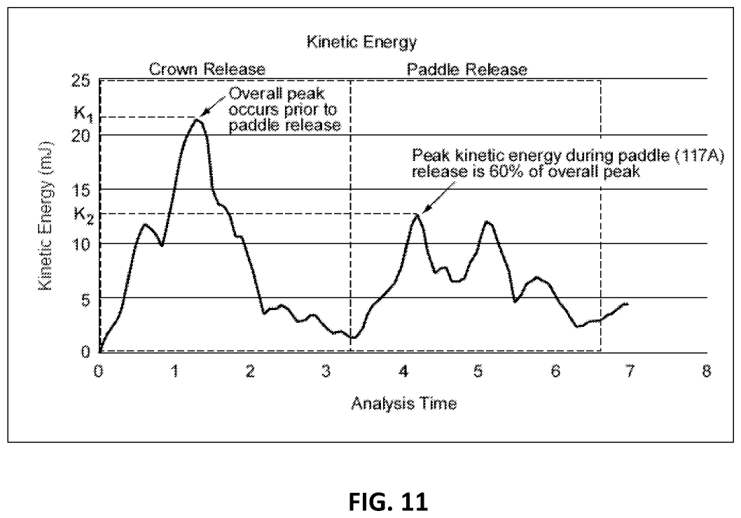

As an example of controlling the kinetic energy of the endcrowns, FIG. 11 provides a graph which depicts the kinetic energy at the endcrowns 103A₁-103Aₙ of the valve prosthesis 101. The graph illustrates the endcrowns 103A₁-103Aₙ having a first level of peak kinetic energy when the endcrowns 103A₁-103Aₙ are released by the capsule 120 but the paddles 117A/117B remain restrained by the capsule 120, and having a second level of peak kinetic energy when the paddles 117A/117B is released and the endcrowns 103A₁-103Aₙ are allowed to fully radially expand. In the example illustrated in FIG. 11, the first level of peak kinetic energy may be in a range of 20 mJ to 25 mJ (e.g., 21 mJ), while the second level of peak kinetic energy may be in a range of 5 mJ to 20 mJ, or more specifically in a range of 10 mJ to 15 mJ (e.g., 12.6 mJ). More particularly, the stem 115A as in the present application may cause the second level of peak kinetic energy associated with expansion of the endmost crowns 103A₁-103Aₙ to the fully deployed configuration to be no more than 60 percent or 70 percent of the first level of peak kinetic energy associated with expansion of the endmost crowns 103A₁-103Aₙ from the radially compressed configuration to the partially deployed configuration. This reduced value of the second level of peak kinetic energy may facilitate accurate deployment of the valve prosthesis 101 by reducing jumping and oscillation as described above.

The foregoing description has been presented for purposes of illustration and enablement and is not intended to be exhaustive or to limit the invention to the precise form disclosed. Other modifications and variations are possible in light of the above teachings. The embodiments and examples were chosen and described in order to best explain the principles of the invention and its practical application and to thereby enable others skilled in the art to best utilize the invention in various embodiments and various modifications as are suited to the particular use contemplated. It is intended that the appended claims be construed to include other alternative embodiments of the invention.

What is claimed is:

1. A valve prosthesis, comprising:
a self-expanding frame including: (i) a plurality of struts forming a plurality of endcrowns at a first end of the self-expanding frame; and (ii) a first paddle connected to a first endcrown of the plurality of endcrowns by a first stem,
wherein the self-expanding frame is:
collapsible to a collapsed configuration at the first end of the self-expanding frame when a restraining force is applied to the first paddle and to the plurality of endcrowns,
configured to expand from the collapsed configuration to a partially deployed configuration when the restraining force is removed from the plurality of endcrowns but is still applied to the first paddle, wherein in the partially deployed configuration the self-expanding frame has a first value of a lateral dimension at the first end, and
configured to expand from the partially deployed configuration to a fully deployed configuration when the restraining force is removed from the plurality of endcrowns and from the first paddle, wherein in the fully deployed configuration the self-expanding frame has a second value of the lateral dimension at the first end, wherein the first value is at least 70 percent of the second value; and
a prosthetic valve component coupled to an interior of the self-expanding frame.

2. The valve prosthesis of claim 1, wherein the first value of the lateral dimension is in a range of 80 percent to 90 percent of the second value of the lateral dimension.

3. The valve prosthesis of claim 1, wherein in the collapsed configuration the plurality of endcrowns store potential energy, and the stored potential energy is reduced by at least 40 percent when the self-expanding frame expands from the collapsed configuration to the partially deployed configuration.

4. The valve prosthesis of claim 3, wherein the stored potential energy is reduced by at least 50 percent when the self-expanding frame expands from the collapsed configuration to the partially deployed configuration.

5. The valve prosthesis of claim 1, wherein a first level of peak kinetic energy is dispersed by the self-expanding frame when expanding from the collapsed configuration to the partially deployed configuration, and a second level of peak kinetic energy is dispersed by the self-expanding frame when expanding from the partially deployed configuration to the fully deployed configuration, and wherein the second level of peak kinetic energy is less than 70 percent of the first level of peak kinetic energy.

6. The valve prosthesis of claim 5, wherein the second level of peak kinetic energy is in a range of 5 milli-Joule (mJ) to 20 mJ.

7. The valve prosthesis of claim 6, wherein the second level of peak kinetic energy is in a range of 10 mJ to 15 mJ.

8. The valve prosthesis of claim 1, wherein the first stem has a length which is at least 4 mm.

9. The valve prosthesis of claim 1, wherein the first stem is formed as a spring having multiple bends, and the spring is stretched when the self-expanding frame expands from the collapsed configuration to the partially deployed configuration.

10. The valve prosthesis of claim 9, wherein the spring has a spring constant which is in a range of 0.2 N/mm to 5 N/mm.

11. The valve prosthesis of claim 1, wherein with the first end in the radially collapsed configuration, the first stem has at least one bend connecting a first section of the first stem and a second section of the first stem, the first section extending from the first endcrown and the second section extending to the first paddle, wherein a gap between the first section and the second section is less than 1 mm.

12. The valve prosthesis of claim 1, further comprising a second paddle connected by a second stem to a second endcrown of the plurality of endcrowns, the first paddle and the second paddle being on opposite sides at the first end of the self-expanding frame.

13. The valve prosthesis of claim 12, wherein the first stem and the second stem have different lengths.

14. The valve prosthesis of claim 12, wherein the first stem and the second stem are about equal in length.
